(19) United States
(12) Patent Application Publication
(10) Pub. No.: US 2020/0268643 A1
(43) Pub. Date: Aug. 27, 2020

(54) ANTIOXIDANT, SKIN-WHITENING AND WRINKLE-REDUCING MULTIFUNCTIONAL COSMETIC COMPOSITION CONTAINING FERMENTED RICE BY-PRODUCT EXTRACT OR FRACTION THEREOF AS ACTIVE INGREDIENT

(71) Applicant: Korea University Research and Business Foundation, Seoul (KR)

(72) Inventors: Seung-Taik LIM, Seoul (KR); Su-Jin OH, Seogwipo-si (KR); Nuntinee RITTHIBUT, Chiang Mai (TH)

(73) Assignee: Korea University Research and Business Foundation, Seoul (KR)

(21) Appl. No.: 16/645,860

(22) PCT Filed: Sep. 21, 2018
(86) PCT No.: PCT/KR2018/011260

§ 371 Date: Mar. 10, 2020

(30) Foreign Application Priority Data
Sep. 26, 2017 (KR) 10-2017-0124489
Sep. 21, 2018 (KR) 10-2018-0113581

(51) Int. Cl. A61K 8/9794; A61K 8/9728; A61Q 19/02; A61Q 19/08

(52) U.S. Cl. CPC A61K 8/9794; A61Q 19/08; A61Q 19/02; A61K 8/9728

(57) ABSTRACT

The present invention relates to an antioxidant, skin-whitening and wrinkle-reducing multifunctional cosmetic composition containing a fermented rice by-product extract or a fraction thereof as an active ingredient. By using a fermented rice by-product extract or a fraction thereof according to the present invention, the total phenolic content of a rice by-product can be increased by means of fermentation, extraction and fractionation, and a cosmetic composition showing high antioxidant capability and excellent skin-whitening and wrinkle-reducing effects can be provided.

### TECHNICAL FIELD

The present invention relates to an antioxidant, skin-whitening and wrinkle-reducing multifunctional cosmetic composition containing fermented rice by-product extract or fraction thereof as active ingredient.

### BACKGROUND ART

Skin aging can be broadly classified into aging caused by intrinsic factors and aging caused by extrinsic factors. Skin aging causes structural and functional alterations in the skin depending on its cause. Most skin age-related changes are affected by external environmental factors such as ultraviolet light because the skin is exposed to the external environment. As aging proceeds, the skin becomes dry, dark, and loses its elasticity, resulting in an increase in the number of wrinkles.

Oxygen free radicals are major causes of aging and are inevitably produced during metabolism where oxygen is required. Very unstable oxygen free radicals attack tissues in the body to oxidize and damage cells, resulting in skin aging.

Oxygen free radicals oxidize proteins in vivo or lipids in cell membranes to deteriorate body functions and damage DNA. Accordingly, oxygen free radicals may also be involved in mutagenesis and carcinogenesis. Further, oxygen free radicals attack collagen and skin fibers to cause skin aging, with the result that skin sagging and laxity are caused to form wrinkles.

The body has enzymatic defense systems such as superoxide dismutase (SOD) against excessive oxygen free radicals. People in their 20s do not have difficulty in keeping their health due to the high activity of their antioxidant enzymatic defense systems. However, since the function of antioxidant enzymes is rapidly attenuated with age, the body is easily exposed to oxygen free radicals and aging is accelerated. Thus, appropriate removal of oxygen free radicals is necessary for anti-aging.

Aging causes different histological changes in skin structures but generally it makes the epidermis thinner, the interface between the epidermis and the dermis flatter, and the number of elastic fibers smaller, leading to the formation of wrinkles.

Skin wrinkling is basically associated with changes of collagen and elastin in the dermal layer as skin aging proceeds with age or under the influence of ultraviolet light. Reduced collagen and elastin in the skin reduce the elasticity of the skin, eventually leading to the formation of wrinkles.

Melanin has a function of protecting the body from cytotoxic agents such as amines, free radicals, and metal ions, but its overproduction leads to skin pigmentation such as melasma and freckles and promotes skin aging.

Melanin is a phenolic polymer widely distributed in nature and is a complex of a black pigment and a protein. The synthesis of melanin starts from the conversion of tyrosine into DOPA in melanocytes. DOPA is then oxidized to DOPA-quinone. Tyrosinase, an oxidase, is involved in the conversion to DOPA-quinone. Thereafter, DOPA-quinone is auto-oxidized to black brown melanin. For this reason, the whitening effect of whitening cosmetics can be expected based on the inhibition of the enzymatic activity of tyrosinase to suppress melanogenesis.

Rice bran refers to a pulverized product of pericarp, testa, and aleurone obtained when unpolished rice is polished into polished rice. It is well known that 95% of the nutrients of rice are present in rice bran and the rice embryo bud and functional ingredients are present in the aleurone layer of unpolished rice. Rice bran contains high quality proteins, dietary fibers, and a variety of vitamins and minerals and is abundant in naturally occurring antioxidants such as bioactive phenolic compounds, flavonoid compounds, γ-oryzanol, γ-aminobutyric acid (GABA), ceramides, tocotrienols, and phytosterols, water soluble vitamins, and minerals such as calcium, phosphorus, and iron.

Despite the presence of various functional ingredients in rice by-products, including rice bran, the majority of rice by-products are wasted or are used only in limited applications such as livestock feed. Rice bran oil has recently been developed in the food industry and technologies for utilizing rice bran in the food and cosmetic industries are currently being developed through studies on various functionalities of rice by-products, including rice bran. However, these technologies are still at the early stage of development.

In this connection, Korean Patent Publication No. 10-2012-0117539 discloses a whitening and anti-wrinkle composition including a rice bran extract. However, the whitening and anti-wrinkle effects of the composition fail to reach satisfactory levels.

Thus, the present inventors have conducted research on the efficacy of rice by-products, including rice bran, and as a result, found that the use of an extract from a fermented rice by-product or a fraction thereof achieves significantly improved antioxidant, skin whitening, and anti-wrinkle effects. The present invention has been accomplished based on this finding.

### DETAILED DESCRIPTION OF THE INVENTION

### Problems to be Solved by the Invention

The present invention has been made in an effort to solve the above problems and intends to provide an antioxidant, skin whitening, and anti-wrinkle multifunctional cosmetic composition including an extract from a fermented rice by-product or a fraction thereof as an active ingredient.

### Means for Solving the Problems

An aspect of the present invention provides an antioxidant, skin whitening, and anti-wrinkle multifunctional cosmetic composition including an extract from a fermented rice by-product or a fraction thereof as an active ingredient.

According to the present invention, the extract from the fermented rice by-product may be prepared by inoculating a rice by-product with at least one fungal species selected from the group consisting of Aspergillus sojae, Aspergillus oryzae, Aspergillus awamori, Aspergillus oryzae, and Monascus pilosus, fermenting the inoculated rice by-product, and extracting the fermented rice by-product.

The fermentation may be performed for 6 to 14 days.

The fermentation may be performed by inoculating 100 parts by weight of the rice by-product with 0.1 to 20 parts by weight of the fungal species and incubating the inoculated rice by-product at 20 to 45° C.

According to the present invention, the fermented rice by-product may be extracted with at least one solvent selected from the group consisting of water, C₁-C₄ anhydrous or hydrous alcohols, ethyl acetate, acetone, glycerin, ethylene glycol, propylene glycol, and butylene glycol.

According to the present invention, the fraction may be prepared by fractionating the extract from the fermented rice by-product with a solvent selected from the group consisting of hexane, chloroform, ethyl acetate, butanol, water, and mixed solvents thereof.

According to the present invention, the composition may be prepared into a formulation selected from the group consisting of solutions, external ointments, creams, foams, nourishing lotions, softening lotions, packs, soft water, emulsions, makeup bases, essences, soaps, liquid cleansers, bath preparations, sunscreen creams, sun oils, suspensions, pastes, gels, lotions, powders, surfactant-containing cleansing agents, oils, powder foundations, emulsion foundations, wax foundations, patches, and sprays.

According to the present invention, the rice by-product may be rice embryo bud or rice bran.

### Effects of the Invention

The use of the extract or fraction thereof prepared by a series of fermentation, extraction, and optional fractionation achieves increased total phenolic content, high antioxidant activity, and significant skin whitening and anti-wrinkle effects of the cosmetic composition according to the present invention.

### BRIEF DESCRIPTION OF THE DRAWINGS

FIG. 1 is a graph showing the total phenolic contents of extracts from rice bran fermented for different times (Example 1).

FIG. 2 is a graph showing the DPPH radical scavenging activities of extracts from rice bran fermented for different times (Example 1).

FIG. 3 is a graph showing the tyrosinase inhibition activities of extracts from rice bran fermented for different times (Example 1), which were measured to investigate the whitening activities of the extracts.

FIG. 4 is a graph showing the elastase inhibition activities of extracts from rice bran fermented for different times (Example 1), which were measured to investigate the anti-wrinkle effects of the extracts.

FIG. 5 shows the results of HPLC for standard compounds at 280 nm, specifically HPLC peaks of (1) gallic acid, (2) 4-hydroxybenzoic acid, and (3) vanillic acid.

FIG. 6 shows the results of HPLC for standard compounds at 320 nm, specifically HPLC peaks of (1) caffeic acid, (2) coumaric acid, (3) sinapic acid, and (4) ferulic acid.

FIG. 7 shows the results of HPLC for (a) an extract from unfermented rice bran and extracts from rice bran fermented for (b) 2, (c) 4, (d) 6, (e) 8, (f) 10, (g) 12, and (h) 14 days (Example 1), which were measured at a UV detection wavelength of 280 nm to identify bioactive substances in the extracts.

FIG. 8 shows the results of HPLC for (a) an extract from unfermented rice bran and extracts from rice bran fermented for (b) 2, (c) 4, (d) 6, (e) 8, (f) 10, (g) 12, and (h) 14 days (Example 1), which were measured at a UV detection wavelength of 320 nm to identify bioactive substances in the extracts.

FIG. 9 shows the results of HPLC for (a) an extract from unfermented rice bran and extracts from rice bran fermented for (b) (b) 2, (c) 4, (d) 6, (e) 8, (f) 10, (g) 12, and (h) 14 days (Example 1), which were measured at a UV detection wavelength of 360 nm to identify bioactive substances in the extracts.

FIG. 10 shows a process for preparing fractions of an extract from a fermented rice by-product in Example 2.

FIG. 11 is a graph showing the tyrosinase inhibition activities of fractions of extracts from fermented rice bran (Example 2), which were measured to investigate the whitening activities of the fractions.

FIG. 12 is a graph showing the elastase inhibition activities of fractions of extracts from fermented rice bran (Example 2), which were measured to investigate the anti-wrinkle effects of the fractions.

### Best Mode for Carrying out the Invention

The present invention will now be described in more detail.

The present invention provides an antioxidant, skin whitening, and anti-wrinkle multifunctional cosmetic composition including an extract from a fermented rice by-product or a fraction thereof as an active ingredient.

The extract from the fermented rice by-product is preferably prepared by inoculating a rice by-product with at least one fungal species selected from the group consisting of Aspergillus sojae, Aspergillus oryzae, Aspergillus awamori, Aspergillus oryzae, and Monascus pilosus, fermenting the inoculated rice by-product, and extracting the fermented rice by-product. As can be particularly seen from the Examples section that follows, extracts from a rice by-product fermented with Aspergillus sojae exhibit markedly improved characteristics in terms of total phenolic content, DPPH radical scavenging activity (DPPH), antioxidant activity, tyrosinase inhibition activity, and elastase inhibition activity.

The fermentation is preferably performed for 6 to 14 days. As can be seen from the Examples section that follows, if the fermentation time is shorter than the lower limit defined above, effects in terms of total phenolic content, DPPH radical scavenging activity (DPPH), antioxidant activity, tyrosinase inhibition activity, and elastase inhibition activity are negligible. Meanwhile, if the fermentation time exceeds the upper limit defined above, the above-described effects are not improved any further.

The fermentation is preferably performed by inoculating 100 parts by weight of the rice by-product with 0.1 to 20 parts by weight of the fungal species and incubating the inoculated rice by-product at 20 to 45° C.

The fermented rice by-product may be extracted with at least one solvent selected from the group consisting of water, C₁-C₄ anhydrous or hydrous alcohols, ethyl acetate, acetone, glycerin, ethylene glycol, propylene glycol, and butylene glycol. The extraction solvent is more preferably selected from water, C₁-C₄ anhydrous or hydrous alcohols, and mixtures thereof. Examples of the C₁-C₄ alcohols include methanol, ethanol, n-propanol, iso-propanol, n-butanol, and tert-butanol. The extraction solvent is most preferably hydrous ethanol in which ethanol is preferably present in an amount of 30 to 90% by volume, more preferably 40 to 70% by volume or hydrous methanol in which methanol is preferably present in an amount of 30 to 90% by volume, more preferably 40 to 70% by volume.

As used herein, the term “fraction” refers to a product obtained by fractionating a mixture including various components to isolate a specific component or a specific group of components from the mixture. The fractionation is not limited to a particular method and may be performed by any suitable method known in the art. According to a non-limiting example, the fraction can be prepared by treating the extract from the fermented rice by-product with a solvent.

The solvent used to obtain the fraction is not limited to a particular kind and may be any of those known in the art. Non-limiting examples of suitable fractionation solvents include: polar solvents, including water and alcohols such as ethanol and butanol; and non-polar solvents such as hexane, ethyl acetate, acetone, and chloroform. These fractionation solvents may be used alone or as a mixture of two or more thereof.

In the Examples section that follows, the fermented rice bran is extracted with 90% ethanol and the extract is stepwise fractionated with hexane, chloroform, ethyl acetate, butanol, and water. Particularly, the hexane and ethyl acetate fractions of the extract from the fermented rice bran have high tyrosinase and elastase inhibition activities compared to the other solvent fractions.

The content of the extract from the fermented rice by-product is from 0.01 to 40% by weight, preferably 0.1 to 10% by weight, based on the total weight of the cosmetic composition according to the present invention. The extract from the fermented rice by-product is in the form of a powder or liquid.

The rice by-products may be rice embryo bud or rice bran.

The cosmetic composition of the present invention may be prepared into a formulation selected from the group consisting of solutions, external ointments, creams, foams, nourishing lotions, softening lotions, packs, soft water, emulsions, makeup bases, essences, soaps, liquid cleansers, bath preparations, sunscreen creams, sun oils, suspensions, pastes, gels, lotions, powders, surfactant-containing cleansing agents, oils, powder foundations, emulsion foundations, wax foundations, patches, and sprays, but is not limited thereto.

The cosmetic composition of the present invention may further include at least one cosmetically acceptable carrier that is blended in general skin cosmetics. Examples of such cosmetically acceptable carriers include, but are not limited to, oils, water, surfactants, moisturizers, lower alcohols, thickeners, chelating agents, pigments, preservatives, and perfumes.

The cosmetically acceptable carrier may vary depending on the formulation of the cosmetic composition.

The formulation may be an ointment, paste, cream or gel. In this case, the carrier may be selected from, but not limited to, animal oils, vegetable oils, waxes, paraffin, starch, tragacanth, cellulose derivatives, polyethylene glycol, silicone, bentonite, silica, talc, zinc oxide, and mixtures thereof.

Alternatively, the formulation may be a powder or spray. In this case, the carrier may be selected from, but not limited to, lactose, talc, silica, aluminum hydroxide, calcium silicate, and polyamide powder. Particularly, when the formulation is a spray, the composition may further include one or more propellants such as chlorofluorohydrocarbons, propane/butane, and dimethyl ether, which may be used alone or as a mixture of two or more thereof.

Alternatively, the formulation may be a solution or emulsion. In this case, the carrier may be selected from, but are not limited to, solvents, solubilizers, and emulsifiers. Examples of suitable carriers include, but are not limited to, water, ethanol, isopropanol, ethyl carbonate, ethyl acetate, benzyl alcohol, benzyl benzoate, propylene glycol or 1,3-butyl glycol oil, particularly, cottonseed oil, peanut oil, corn germ oil, olive oil, castor oil, sesame oil, glycerol aliphatic esters, polyethylene glycol, and sorbitan fatty acid esters. These carriers may be used alone or as a mixture of two or more thereof.

Alternatively, the formulation may be a suspension. In this case, the carrier may be selected from, but not limited to, liquid diluents such as water, ethanol, and propylene glycol, suspending agents such as ethoxylated isostearyl alcohol, polyoxyethylene sorbitol esters, and polyoxyethylene sorbitan esters, microcrystalline cellulose, aluminum metahydroxide, bentonite, agar, and tragacanth. These carriers may be used alone or as a mixture of two or more thereof.

Alternatively, the formulation may be a soap. In this case, the carrier may be selected from, but not limited to, alkali metal salts of fatty acids, hemi-ester salts of fatty acids, fatty acid protein hydrolysates, isethionates, lanolin derivatives, aliphatic alcohols, vegetable oils, glycerol, and sugars. These carriers may be used alone or as a mixture of two or more thereof.

### MODE FOR CARRYING OUT THE INVENTION

The present invention will be explained in more detail with reference to the following examples. It will be evident to those skilled in the art that these examples are not intended to limit the scope of the present invention.

### EXAMPLE 1

### Preparation of Extract from Fermented Rice Bran

(1) Preparation of Fermented Rice Bran

Aspergillus sojae KCCM 60354 was cultured in potato dextrose agar (PDA) until sporulation occurred (day 14). The spores were collected, washed twice, and stored at 80° C. for further use. Rice bran was mixed with water in a ratio of 1:1 (w/w) and sterilized using an autoclave at 121° C. for 20 min. The sterilized rice bran was transferred to a Petri dish. 1 part by weight of the fungal spores (1 ml spore solution (1 ×10₆ spores/ml)) were sufficiently mixed with 100 parts by weight of the rice bran (100 g of the rice bran). The mixture was incubated at 25° C. for 14 days. Samples were collected every 2 day during incubation and stored at 20° C. for further analysis.

### (2) Preparation of Extracts From the Fermented Rice Bran

Each of the fermented rice bran samples collected at different incubation times was extracted with 95% ethanol, shaken at 100 rpm and 25° C. for 24 h, filtered, and centrifuged at 3500 rpm for 15 min. This extraction procedure was repeated twice. The supernatant was collected, evaporated, and lyophilized to prepare an extract from the fermented rice bran.

### EXAMPLE 2

### Preparation of Fractions of the Extracts from the Fermented Rice Bran

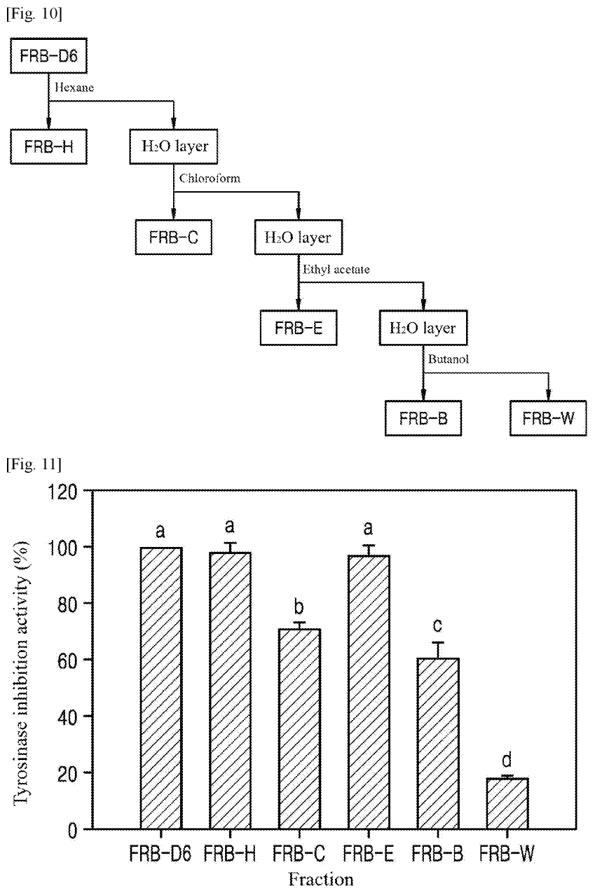

On day 6 of fermentation, the ethanol extract from the rice bran (FRB-D6: 95% ethanol) was stepwise fractionated with hexane, chloroform, ethyl acetate, butanol, and water in a ratio of 1:10 (v/v) to isolate active ingredients (FIG. 10). The hexane (FRB-H), chloroform (FRB-C), ethyl acetate (FRB-E), butanol (FRB-B), and water (FRB-W) fractions were collected, evaporated, and lyophilized for further experiments.

### EXPERIMENTAL EXAMPLE 1

### Total Phenolic Content (TPC) Measurement

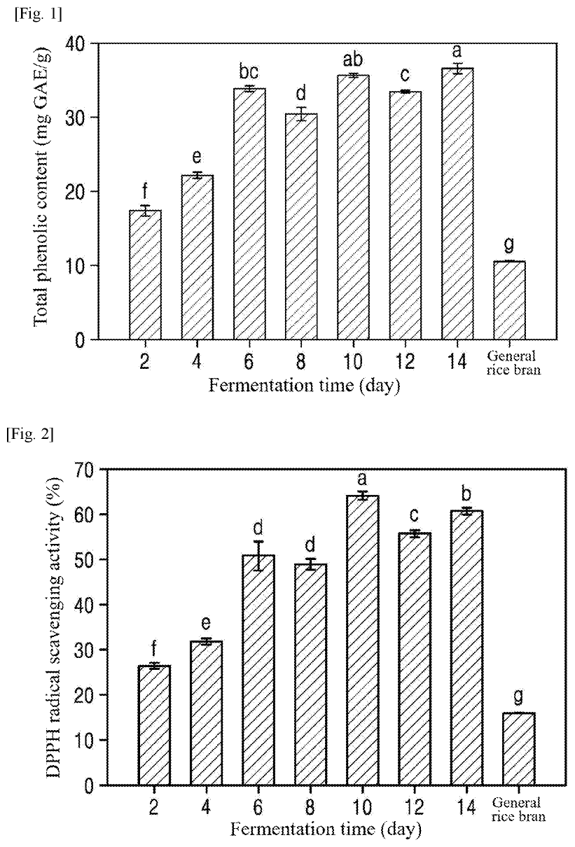

The total phenolic contents (TPCs) of the extracts from the fermented rice bran were measured by the modified Ainsworth and Gillespie method. Specifically, 200 μl of 10% (v/v) Folin-Ciocalteu reagent and 800 μl of 700 mM Na₂CO₃ were mixed with 100 μl of each of the extracts from the fermented rice bran. The mixture was allowed to stand at room temperature for 2 h. Thereafter, the absorbance of the mixture was measured at 765 nm using a spectrophotometer. The TPC was calculated using the gallic acid standard curve. The results are shown in FIG. 1.

As shown in FIG. 1, all extracts from the rice bran fermented by Aspergillus sojae showed higher total phenolic contents than general rice bran. The total phenolic content tended to increase with increasing fermentation time. Particularly, the total phenolic content of the extract from the rice bran fermented for 14 days (36.5±0.7 mgGAE/g) was found to be ˜3 times higher than that of general rice bran (10.6±0.1 mgGAE/g).

### EXPERIMENTAL EXAMPLE 2

### Antioxidant Activity Measurement

The antioxidant activity of each of the extracts from the fermented rice bran was observed by measuring the 2, 2-diphenyl-1-picrylhydrazyl (DPPH) radical scavenging activity of the extract. Specifically, 450 μl of a 100 mM Tris-HCl buffer (pH 7.4) and 1 ml of a 0.1 mM DPPH solution were mixed with 50 μl of each of the extracts from the fermented rice bran. The mixture was allowed to stand in the dark at room temperature for 30 min. Thereafter, the absorbance of the mixture was read at 517 nm using a spectrophotometer. The DPPH radical scavenging activity (%) was calculated using Equation 1:

DPPH radical scavenging activity(%)=(Absorbance of blank sample−(Absorbance of sample/Absorbance of blank sample)×100 (1)

The results are shown in FIG. 2. As shown in FIG. 2, the DPPH radical scavenging activities of the extracts from the rice bran fermented by Aspergillus sojae were improved compared to that of general rice bran. Particularly, the DPPH radical scavenging activity of the extract from the rice bran fermented for 10 days (63.9 ±0.9%) was ˜4 times higher than that of general rice bran (15.7 ±0.2%).

### EXPERIMENTAL EXAMPLE 3

### Whitening Activity Measurement

(1) The whitening activity of each of the extracts from the fermented rice bran was observed by measuring the tyrosinase inhibition activity of the extract. Specifically, 40 μl of each of the extracts from the fermented rice bran was dissolved in 40 pl of tyrosinase (240 U/ml) at pH 6.5, mixed with 5% DMSO and 80 μl of a 50 mM potassium phosphate buffer (P-buffer), incubated at 25° C. for 10 min, and added with 40 μl of 0.85 mM L-3,4-dihydroxyphenylalanine (L-DOPA). Next, the mixture was continuously incubated at 25° C. for 5 min and measured for absorbance at 490 nm using a microplate spectrophotometer (Bio-Tek). The tyrosinase inhibition activity (%) was calculated using Equation 2:

Tyrosinase inhibition activity(%)=(((A−B)−(C−D))÷(A−B))×100 (2)

where A=Absorbance of blank sample containing tyrosinase

B=Absorbance of blank sample without tyrosinase

C=Absorbance of sample containing tyrosinase

D=Absorbance of sample without tyrosinase

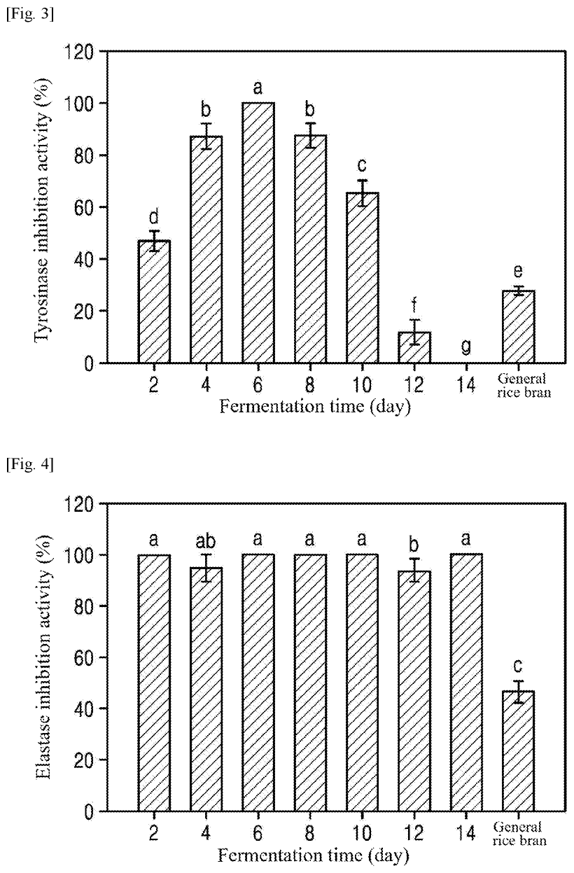

The results are shown in FIG. 3. As shown in FIG. 3, the tyrosinase inhibition activity of general rice bran was as very low as ˜27.8%, while that of each of the extracts from the rice bran fermented for 2-10 days was improved. Particularly, the tyrosinase inhibition activity of the extract from the rice bran fermented for 6 days was ˜100%, which was ˜3.6-fold higher than that of general rice bran (IC₂₀=1.55 mg/ml).

(2) On day 6 of fermentation, the whitening activity of each of the fractions of the ethanol extracts from the rice bran (FRB-D6: 95% ethanol) was observed by measuring the tyrosinase inhibition activity of the fraction. The experimental procedure was the same as explained in (1). The results are shown in FIG. 11 and Table 1.

TABLE 1

| Fraction | IC₅₀ (mg/ml) |
|---|---|
| FRB-D6 | 1.55 |
| FRB-H | 1.27 |
| FRB-C | 5.56 |
| FRB-E | 0.59 |
| FRB-B | — |
| FRB-W | — |

As shown in FIG. 11, the tyrosinase inhibition activities of FRB-D6, FRB-H, and FRB-E were found to be very high (≥˜97%). However, the IC₅₀ values of FRB- D6,FRB-H, and FRB-E were significantly different, as shown in Table 1 . Particularly, the IC₅₀ of FRB-E was 0.59 mg/ml, which was 2.6-fold lower than that of the crude extract (FRB-D6).

### EXPERIMENTAL EXAMPLE 4

### Anti-Wrinkle Activity Measurement

(1) The anti-wrinkle activity of each of the extracts from the fermented rice bran was observed by measuring the elastase inhibition activity of the extract. Specifically, 100 μl of a 2 mM Tris buffer (pH 8.0) and 10 μl of elastase (1.1 U/ml) were mixed with 30 μl of each of the extracts from the fermented rice bran, incubated at 25° C. for 10 min, and added with 40 μl of 3 mg/ml N-Succ-Ala-Ala-Ala-p-nitroanilide (SANA). The mixture was continuously incubated at 25° C. for 20 min and measured for absorbance at 410 nm using a microplate spectrophotometer (Bio-Tek). The elastase inhibition activity (%) was calculated using Equation 3:

Elastase inhibition activity(%)=(((A−B)−(C−D))÷(A−B))×100 (3)

where A=Absorbance of blank sample containing elastase

B=Absorbance of blank sample without elastase

C=Absorbance of sample containing elastase

D=Absorbance of sample without elastase

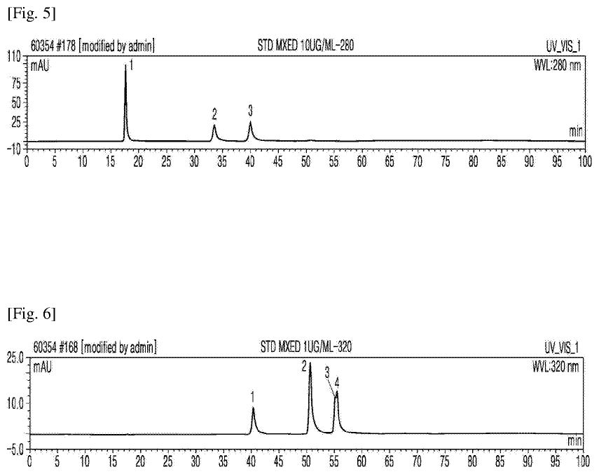

The results are shown in FIG. 4. As shown in FIG. 4, the elastase inhibition activities of the extracts from the rice bran fermented by Aspergillus sojae were ≥90% and were higher than that of general rice bran (˜42%). Particularly, the extract from the rice bran fermented for 6 days showed the highest elastase inhibition activity and was measured to have an IC₅₀ of 3.70 mg/ml.

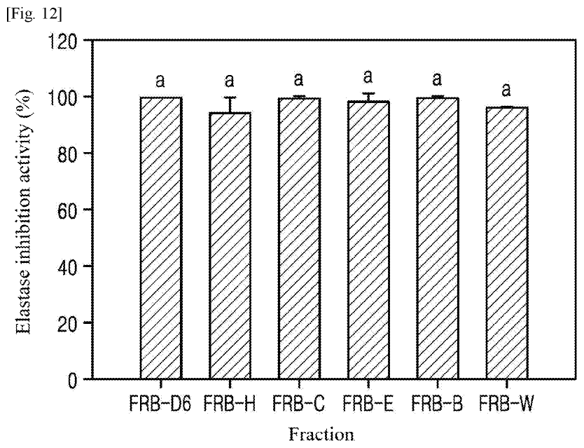

(2) On day 6 of fermentation, the anti-wrinkle activity of each of the fractions of the ethanol extracts from the rice bran (FRB-D6: 95% ethanol) fermented for 6 days was observed by measuring the elastase inhibition activity of the fraction. The experimental procedure was the same as explained in (1). The results are shown in FIG. 12 and Table 2.

TABLE 2

| Fraction | IC₅₀ (mg/ml) |
|---|---|
| FRB-D6 | 3.7 |
| FRB-H | 0.11 |
| FRB-C | 1.34 |
| FRB-E | 0.12 |
| FRB-B | 0.67 |
| FRB-W | 2.43 |

As shown in FIG. 12, the elastase inhibition activities of all fractions except FRB-H were very high (≥94%). However, the IC₅₀ values of the fractions were significantly different, as shown in Table 2 . Particularly, the IC₅₀ of FRB-H was found to be the lowest (0.11 mg/ml) and the IC₅₀ of FRB-E was 0.12 mg/ml, which was 33-fold lower than that of the crude extract (FRB-D6).

### EXPERIMENTAL EXAMPLE 5

### Identification of Bioactive Substances

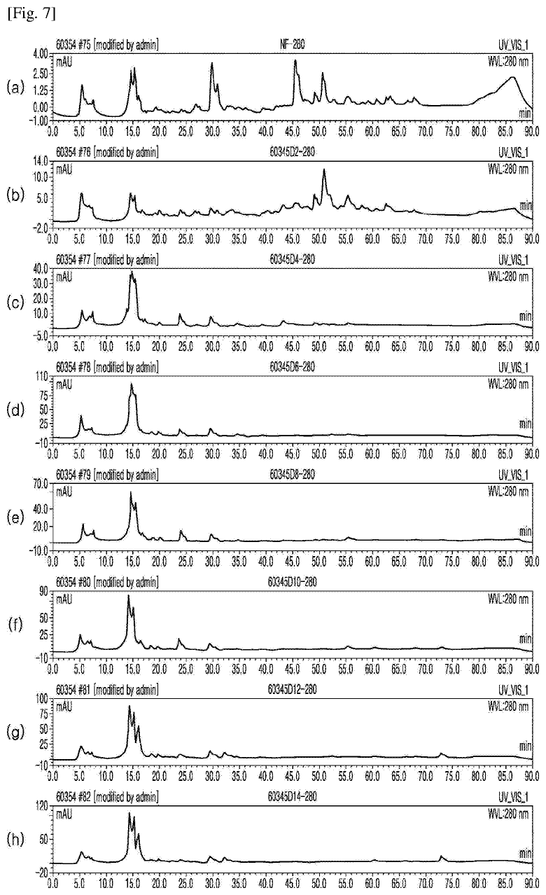

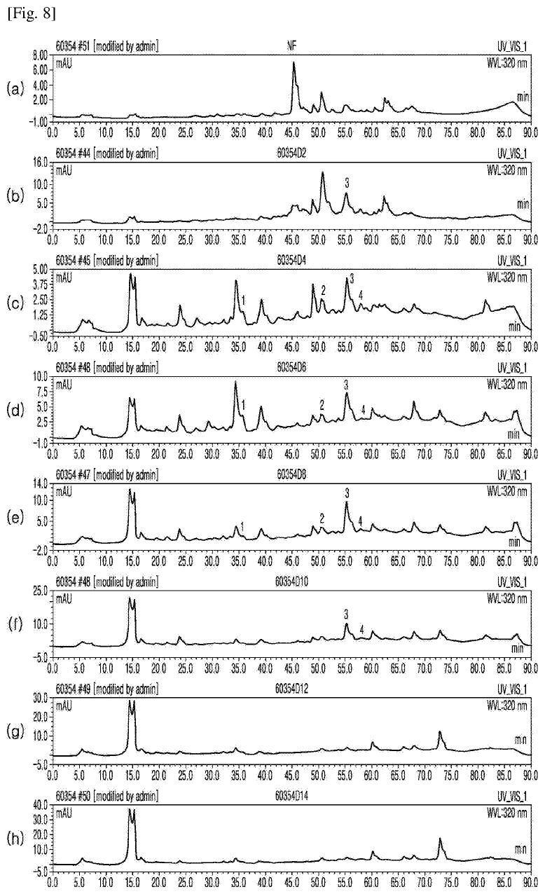

The profiles of bioactive substances in the extracts from the rice bran fermented by Aspergillus sojae were measured by HPLC. Specifically, each of the extracts from the fermented rice bran was filtered through a 0.2 μl filter and HPLC was performed using DIONEX UltiMate 3000 (Thermo Scientific, CA, USA) and a reverse-phase Acclaim TM 120 C18 (5 μl) column (4.6×250 mm, Thermo Scientific, CA, USA). The mobile phase was a gradient mixture of 0.05% phosphoric acid (Solution A) and acetonitrile (Solution B). The gradient program was as follows: 0 min, 0% B; 10 min, 7% B; 20 min, 10% B; 30 min, 12% B; 50 min, 23% B; 70 min, 35% B; 80 min, 50% B; 90 min, 0% B. The flow rate was 0.5 ml/min and the temperature was set to 40° C. Spectra were measured at UV detection wavelengths of 280, 320, and 360 nm using a UV-Vis detector. The results are shown in FIGS. 5-9.

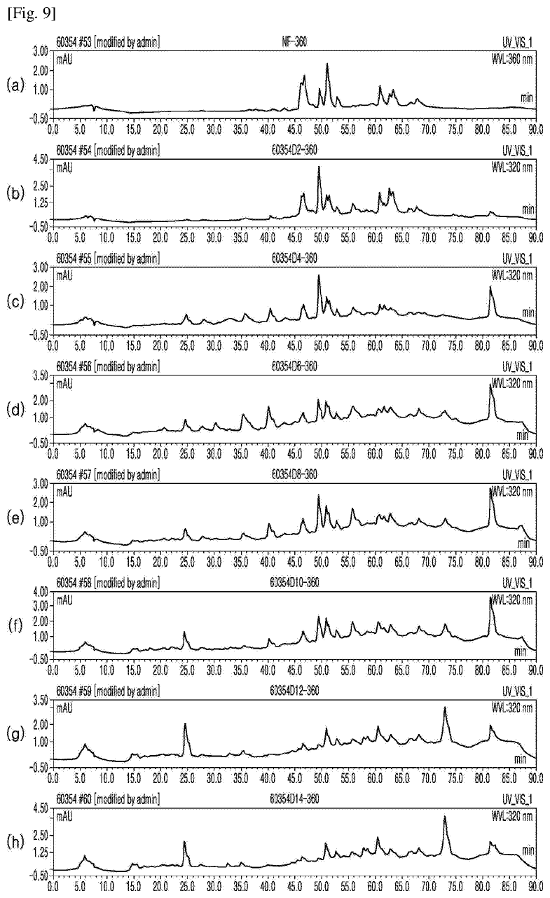

As shown in FIGS. 5-7, gallic acid, 4-hydroxybenzoic acid, and vanillic acid began to be detected at a UV detection wavelength (UV) of 280 nm from 6 days after fermentation. As shown in FIGS. 6 and 8, phenolic acids (caffeic acid, coumaric acid, sinapic acid, and ferulic acid) were detected at a UV detection wavelength of 320 nm. As shown in FIG. 9, various flavonoid compounds were detected at a UV detection wavelength (UV) of 360 nm.

### INDUSTRIAL APPLICABILITY

The composition of the present invention is suitable for use in cosmetic products due to its high antioxidant activity and significant skin whitening and anti-wrinkle effects.

1. An antioxidant, skin whitening, and anti-wrinkle multifunctional cosmetic composition comprising an extract from a fermented rice by-product or a fraction thereof as an active ingredient.

2. The antioxidant, skin whitening, and anti-wrinkle multifunctional cosmetic composition according to claim 1, wherein the extract from the fermented rice by-product is prepared by inoculating a rice by-product with at least one fungal species selected from the group consisting of Aspergillus sojae, Aspergillus oryzae, Aspergillus awamori, Aspergillus oryzae, and Monascus pilosus, fermenting the inoculated rice by-product, and extracting the fermented rice by-product.

3. The antioxidant, skin whitening, and anti-wrinkle multifunctional cosmetic composition according to claim 2, wherein the fermentation is performed for 6 to 14 days.

4. The antioxidant, skin whitening, and anti-wrinkle multifunctional cosmetic composition according to claim 2, wherein the fermentation is performed by inoculating 100 parts by weight of the rice by-product with 0.1 to 20 parts by weight of the fungal species and incubating the inoculated rice by-product at 20 to 45° C.

5. The antioxidant, skin whitening, and anti-wrinkle multifunctional cosmetic composition according to claim 1, wherein the fermented rice by-product is extracted with at least one solvent selected from the group consisting of water, C₁-C₄ anhydrous or hydrous alcohols, ethyl acetate, acetone, glycerin, ethylene glycol, propylene glycol, and butylene glycol.

6. The antioxidant, skin whitening, and anti-wrinkle multifunctional cosmetic composition according to claim 1, wherein the fraction is prepared by fractionating the extract from the fermented rice by-product with a solvent selected from the group consisting of hexane, chloroform, ethyl acetate, butanol, water, and mixed solvents thereof.

7. The antioxidant, skin whitening, and anti-wrinkle multifunctional cosmetic composition according to claim 1, wherein the composition is prepared into a formulation selected from the group consisting of solutions, external ointments, creams, foams, nourishing lotions, softening lotions, packs, soft water, emulsions, makeup bases, essences, soaps, liquid cleansers, bath preparations, sunscreen creams, sun oils, suspensions, pastes, gels, lotions, powders, surfactant-containing cleansing agents, oils, powder foundations, emulsion foundations, wax foundations, patches, and sprays.

8. The antioxidant, skin whitening, and anti-wrinkle multifunctional cosmetic composition according to claim 1, wherein the rice by-product is rice embryo bud or rice bran.
